(19) United States
(12) Patent Application Publication
(10) Pub. No.: US 2016/0157748 A1
(43) Pub. Date: Jun. 9, 2016

(54) Probe for obtaining bioelectrical signals

(71) Applicant: Phillip Dietz, Saint George, UT (US)

(72) Inventor: Phillip Dietz, Saint George, UT (US)

(21) Appl. No.: 14/269,253

(22) Filed: May 5, 2014

(51) Int. Cl. A61B 5/053; A61H 39/02

(52) U.S. Cl. CPC A61B 5/0532; A61H 39/02

(57) ABSTRACT

The present invention is a device for obtaining an electrical signal from a patient that corresponds to a meridian. One embodiment of the present invention comprises an ergonomically pistol grip device that reduces fatigue by the placement of the head and motor and other key components. Another embodiment of the invention is the location of a fan and sound-deadening foam to limit the noise and heat to which the user is exposed during operation of the probe. Embodiment is the isolation hood which contains a removable tip and ergonomically design to prevent user fatigue and comfort to the patient.

The present invention relates to a probe for obtaining electrical signals from a patient to assess a medical condition. In particular, the present invention relates to a probe for accurately locating a meridian transdermally and obtaining a value for an electrical attribute that is ergonomically design and easy to manufacture.

### BACKGROUND

### Prior Art

Traditional medical science has long recognized certain electrical characteristics of humans and other living organisms. For example, the traditional medical community has recognized electrical potentials generated by the human body in such forms as brain waves, detected by electro-encephalographs (EEG), electrical impulses resulting from muscular heart activity, as detected by electrocardiograms (EKG), and other electrical potentials measurable at other areas of the human body. While the levels of electrical activity at sites on the human body are relatively small, such signals are nonetheless measurable and consistent across the species.

In addition to measurable currents, the human body and other mammalian organisms exhibit specific locations where a resistance value and, inversely, a conductance value are relatively predictable for healthy individuals. These locations, known as anatomical dermal conductance points, exhibit unique resistance values. Interestingly, such locations exhibit a resistive reading of approximately 100,000 ohms and coincide with the acupuncture points defined anciently by 35 the Chinese.

Ancient Chinese medical practitioners treated many unfavorable health conditions by inserting thin needles into the body at specific points to pierce peripheral nerves, a technique commonly known as acupuncture. Acupressure is a gentle, noninvasive form of the ancient Chinese practice of acupuncture that implements thumb or finger pressure or electrical stimulation at these same points, also known as acupressure points, to provide similar results.

The representative acupressure points and their relationship with organs and life systems of the human body have been characterized into more than 800 points that are organized into approximately 12 basic meridians that run along each side of the body. Each pair of meridians corresponds to a specific organ or function such as stomach, liver, spleen, pancreas and lung. Acupressure points are named for the meridian they lie on, and each is given a number according to where along the meridian it falls. For example, Spleen 6 is the sixth point on the Spleen meridian. The measurable attributes of each acupressure point reflect the energetic condition of the inner organ or other functions of the human body corresponding to such point.

Acupressure points are generally located at the extremity region of the hands and feet. As introduced above, the resistance value of healthy tissue measured at an acupressure point is generally in the range of about 100,000 ohms. When conditions arise affecting higher conductivity readings, perhaps from inflammation or infection, the measured resistance value becomes less than 100,000 ohms. Likewise when conditions arise affecting lower conductivity readings, perhaps from tissue fatigue or a degenerative state, conductivity is reduced, causing the resistance value to be higher.

Systems have been implemented to measure a resistance, voltage, and/or current values at acupressure points located on a meridian and to present the values to a clinician for use in assessing a condition. Traditional systems, however, have proven difficult to use in pinpointing the precise location of such acupressure points, as required to effectively assess a medical condition. Indeed, most known systems require contacting an acupressure point with the probe tip placed with a specific amount of pressure at a specific angle to obtain a reliable electrical measurement for assessment use.

Measurement inaccuracies result from the failure to precisely locate the probe tip on the acupressure point and properly apply the appropriate rate and amount of pressure to the probe tip. Furthermore, if too much or too little pressure is applied to the point or if the pressure is applied too slowly or too quickly the measured values will either be false high or low.

Learning the proper techniques to obtain accurate readings can take months and even then some may not ever be able to acquire the skill necessary to respectively obtain accurate readings. One of the reason why learning proper techniques takes so long is the prior art probes are difficult to use. The prior art probes caused the users hand to become easily fatigue, the probes are noisy and difficult to manufacture. Prior inventions such as the Horne U.S. Pat. No. 7,542,796 teaches an improved method for obtaining the electrical signal. However, the prior art probe and the probe taught in Horne, have the same limitations stated above.

Accordingly, what is needed is an improved probe for accurately applying the appropriate rate and amount of pressure to the probe tip and locating a meridian or acupressure point that limits hand fatigue, limits noise, and is easier to manufacture.

### SUMMARY OF THE INVENTION

The present invention is a probe for obtaining measured electrical values from a patient that correspond to a meridian or acupressure point. The present invention contemplates an agronomical probe for measuring and comparing the conductivity of a patient's skin. An isolation hood at the end of the probe is then held in contact with the dermal area, and the probe tip is directed toward the skin by a motor actuated to obtain an electrical signal there from. The probe is specially designed with a motor and logic feedback loop to apply an appropriate amount of pressure to the dermal area to accurately measure the signal at a meridian. In addition, to provide a probe that is easily manufacture.

An object of some embodiments is to minimize the electrical interference from the motor on readings from the probe tip. Yet another object of some embodiments of the present invention is to provide a method for obtaining an electrical signal from a patient that enables fast and accurate results that limits users hand fatigue, limits the noise, and protects the users from the heat produced by the motor.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DESCRIPTION OF THE DRAWINGS

The invention may take form in certain parts and arrangement of parts, and preferred embodiments of which will be described in detail in the specification and illustrated in the accompany drawing, which for a part hereof:

FIG. 1 shows a side plan view of the probe;

FIG. 2 shows a cross section of the probe illustrating the location of the motor and the location of the electrical cords;

FIG. 3 shows a cross section of the probe illustrating the location of the fan and the sound deadening foam;

FIG. 4 shows a side view with the exploded view of the tip and the isolation hood;

FIG. 5 shows a cross section of the probe illustrating; and

FIG. 6 shows a side plan and the longitudinal axis of the handle and head.

Drawing -Reference Numbers

| | |
|---|---|
| 2 | Probe |
| 4 | Cord |
| 6 | Trigger |
| 8 | Handle |
| 10 | Isolation hood |
| 12 | Head |
| 13 | Proximal end |
| 14 | Air vent |
| 15 | Distal end |
| 16 | Fan |
| 17 | Concave surface |
| 18 | Sound-deadening foam |
| 20 | Motor |
| 21 | Removable cover |
| 22 | Washer |
| 26 | Detector |
| 28 | Probe tip |
| 30 | O-ring |
| 32 | Wire |
| 34 | Locking screw |
| 36 | Wire Stay |

### DETAILED DESCRIPTION OF THE PREFERRED EMBODIMENTS

The following discussion describes embodiments of the invention and several variations of these embodiments. This discussion should not be construed, however, as limiting the invention to these particular embodiments. Practitioners skilled in the art will recognize numerous other embodiments as well. It is not necessary that the probe have all the feature described below with regard to the specific embodiment of the invention shown in the figures.

In the flowing description of the invention, certain terminology is used for the purpose of reference only, and is not intend to be limiting. Terms such as “upper”, “lower”, “above”, and “below,” refer to directions in the drawings to which reference is made. Terms such as “inward” and “outward” refer to directions towards and away from, respectively, the geometric center of the component described. Terms such as “side”, “top”, “bottom,” “horizontal,” and “vertical,” describe the orientation of portions of the component within a consistent but arbitrary frame of reference which is made clear by reference to the text and the associated drawings describing the component under discussion. Such terminology includes words specifically mentioned above, derivatives thereof, and words of similar import.

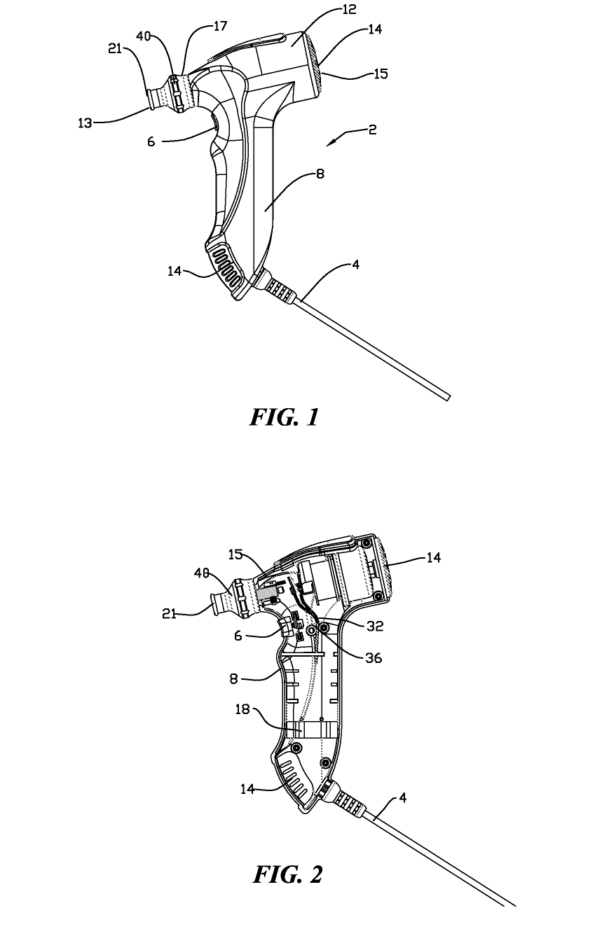

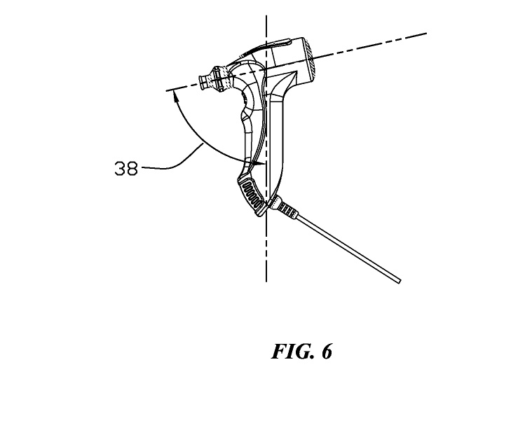

Referring to FIGS. 1 and 6 shows certain embodiments of the present device. A probe 2 assembly comprises a handle 8 and a head 12. In practice, the head 12 and the handle 8 are formed as a single piece made from the same material such as plastic. The longitudinal axis of the handle 8 is generally perpendicular to the longitudinal axis of the head 12, thus forming a pistol grip. As shown in FIG. 6, the preferred embodiment of the invention is the longitudinal axis of the head 12 is at a 85 to 75 degree angle 38 to the longitudinal axis of the handle 8. This angle allows for the probe 2 to fit easily into a user's hand.

The head 12 comprises a proximal end 13 and a distal end 15. Located at the proximal end 13 is an isolation hood 40. Located at the distal end 15 is an air vent 14. The air vent 14 allows for the flow of air through the head 12. Contained inside the head 12 near the proximal end 13 is a probe tip 28. The probe tip 28 is functionally discrete from the isolation hood 10. In this manner, pressure applied to the isolation hood 10 does not affect pressure applied to the probe tip 28. The isolation hood 10 allows the probe tip 28 to independently slide through the isolation hood 10. The isolation hood 10 thus ensures that an electrical signal obtained by the probe tip 28 is objective and repeatable by preventing manipulation of the probe 2.

The center of the isolation hood 10 flairs outwards to create a concave surface 17. The concave surface 17 allows the user to easily grasp the isolation hood 10. In operation of the probe 2, the user would place their thumb and index finger on the concave surface 17. The isolation hood 10 is mainly comprised of stainless steel. However, the isolation hood 10 may be composed of other non-corrosive materials, such as ceramics or plastics. A rubberized material may be placed on the outside surface of the isolation hood 10 to increase the friction between the user's fingers and the probe 2. Located at the end of the isolation hood 40 is a removable cover 21. During use of the probe, the removable cover 21 is placed against the patient's skin. The removable cover 21 is removed and placed after each patient. The removable cover 21 is made with any material that is comfortable to the patient such as silicone or a soft plastic.

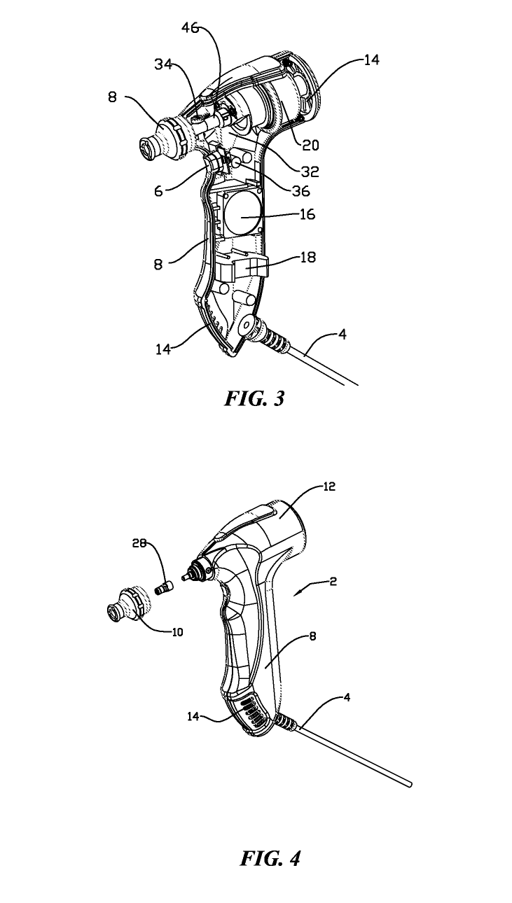

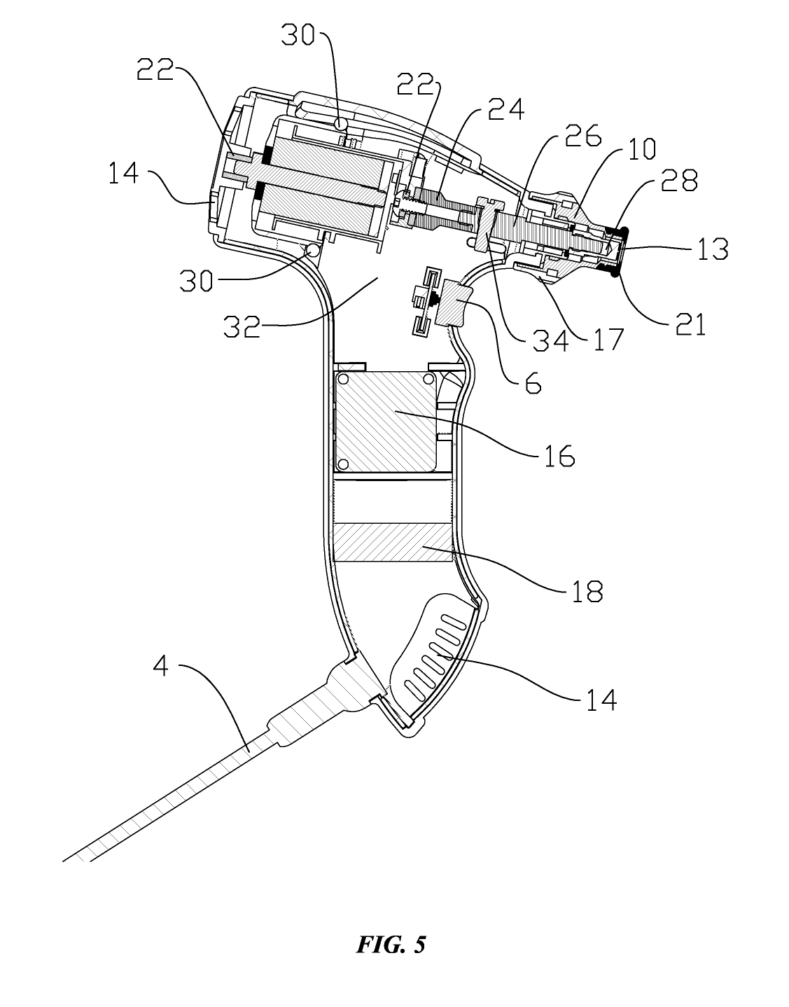

As illustrated in FIGS. 3 and 5, located near the distal end 15, is a motor 20, which is enclosed within in the head 12. An O-ring 30 is placed between the head 8 and the motor 20. This assembly prevents the movement of the motor 20 within the head 8 and the heat from the motor 20 transferring to the head 12. The O-ring 30 may be made of any rubberized heat resistant material such as silicone.

Coupled to the probe tip 28 is a detector 26. The connection between the probe tip 28 and detector 26, is such that the user may easily but purposely remove the probe tip 28. The preferred method of connection? is screw threads. The detector 26 is made of any material that conducts an electrical signal from the probe tip 28 to a wire 32. A locking screw 34 prevents the rotation of the detector 26. The locking screw 34 also allows for the attachment of the wire 32 to the detector 26.

As shown in FIG. 5, an isolator 46 connects the motor 20 to the detector 26. The isolator 46 is made of any material that insulates electrical interference from the motor 20 to the isolator 46. The portion of the isolator 14 connected to the motor 20 has a larger diameter than the portion of the isolator 14 connected to the detector 26. This concentricity allows for an easier transferring movement from the motor 20 to the detector 26 and is easier in manufacturing the probe 2. To future insulate the motor 20 and to prevent heat to be transferred from the motor 20, nonconductive heat resistant washers 22 are located between the isolator 14 and the motor 20. The preferred material of the washer 22 is ceramic.

The handle 8 comprises a trigger 6, a fan 16, and a sound-deadening foam 18. The fan 16 draws air through the air vent 14 located on the distal end 15 of the head 12. The air is drawn around the motor 20, thus cooling the motor 20. The fan continues to draw air through the handle 8 and discharges the air through the bottom of the handle 8 through the air vents 14. The sound-deadening foam 18 deadens the noise from the motor 20. Furthermore, the sound-deadening foam 18 material secures the wires 32 in the handle 8. The sound-deadening foam may be made from any noise deadening material. Located at the base of the handle 8 is a cord 4. The cord 4 supplies power to the probe 2 and provide feedback to a device to measure the bioelectrical signals from the probe 2.

As illustrated in FIGS. 1, 2, 3, and 5, the trigger 6 is located at the top of the handle 8. The preferred location of the trigger 6 is where the user may use their index or middle finger to press the trigger 6. When the trigger 6 is activated, this allows for electrical power to flow to the motor 20 and to activate a reading from the probe tip 28.

Located at the junction head 12 and handle 8 is a cavity 32. The cavity 32 allows the wires 32 connected to the detector 26 to move freely when the detector 26 moves. To secure wires 32 from moving in the handle, a wire stay 36 is located at the base of the cavity 32.

While a preferred embodiment of the invention of the probe 2 has been shown and described herein, it should, however, be understood that the description above contains many specificities that should not be construed as limiting the scope of the invention. Thus, the scope of the embodiment should be determined by the appended claims and their legal equivalents thereof, rather than by the examples given.

What is claimed:

1. A device for obtaining an electrical signal from a patient at the patient's skin, said device comprising:
(a) a probe containing a head and a handle;
wherein the handle connects to the head to form a pistol grip;
(b) a trigger;
(c) a probe tip connected to a motor;
(d) an isolation hood;
wherein the probe tip and the motor are located within the head;
the isolation hood is located on the head;

2. The device of claim 1, wherein said handle longitudinal axis is angle 10 to 20 degrees from the longitudinal axis of the handle.

3. The device of claim 1, wherein said motor is connected to the head by an O-ring and at least one ceramic washer.

4. The device of claim 3, wherein said O-ring is made of heat resistance silicon.

5. The device of claim 1, wherein said isolation hood is made of stainless steel.

6. The device of claim 1, wherein said handle contains a fan;
wherein the fan draws air through the probe.

7. The device of claim 1, wherein said handle contains a sound-deadening foam
wherein the sound-deadening foam limits the noise from the probe.

8. The device of claim 1, wherein at the base of said handle is a cord;
wherein the cord provides electrical power to the probe.

9. The device of claim 1, wherein said motor and said probe tip are coupled by an isolator.

10. The device of claim 9, wherein said isolator is non-conductive.

11. The device of claim 1, wherein said isolation hood is made of stainless steel.

12. The device of claim 1, wherein said isolation hood has a concave surface;

13. The device of claim 1, wherein said isolation hood has a removable cover;
wherein said removable cover is placed against a dermal surface.

14. A method for obtaining an electrical signal from a patient at the patient's skin, said method comprising;
(a) providing a device includes;
(i) a probe containing a head and a handle;
wherein the handle connects to the head to form a pistol grip;
(ii) a trigger;
(iii) a probe tip connected to a motor;
(iv) an isolation hood;
wherein the probe tip and the motor are located within the head;
the isolation hood is located on the head;
(b) placing the isolation hood against a patient's skin;
(c) obtaining for said probe an electrical signal at the patient's skin

15. The method of claim 14, wherein said, wherein said isolation hood comprises a removable cover,
wherein, said removable cover and the probe tip are replaced after each patient.

16. The method of claim 14, wherein in said motor's electrical interference and heat is insulated from said probe by an o-ring and a washer.

17. The method of claim 16, wherein said O-ring is made of a silicon material and said washer is made from ceramic material.
